(19) United States
(12) Patent Application Publication
(10) Pub. No.: US 2024/0099215 A1
(43) Pub. Date: Mar. 28, 2024

(54) PHILODENDRON WHITE PRINCESS PLANT NAMED "White Ice Philodendron"

(71) Applicant: Natalie Roe, Umatilla, FL (US)

(72) Inventor: Natalie Roe, Umatilla, FL (US)

(21) Appl. No.: 17/953,985

(22) Filed: Sep. 27, 2022

(51) Int. Cl. A01H 6/10; A01H 5/12

(52) U.S. Cl. CPC A01H 6/10; A01H 5/12

(57) ABSTRACT

A new and distinct cultivar of Philodendron White Princess named “White Ice Philodendron” particularly characterized by its distinctive leaf appearance; large plant size; mostly open and outwardly spreading fuller plant habit; vigor and slow growth rate; less-freely branching; flat leaves; and long and wide petioles with long petiole sheaths.

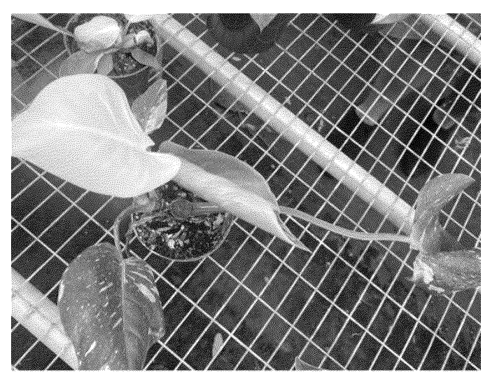

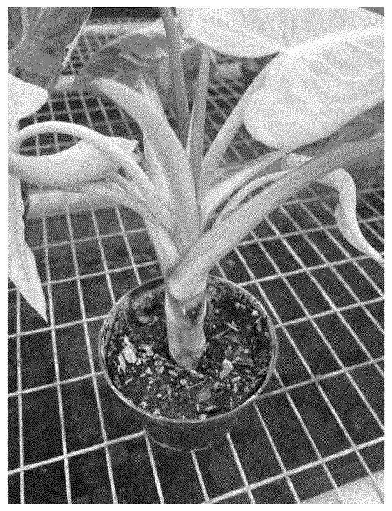

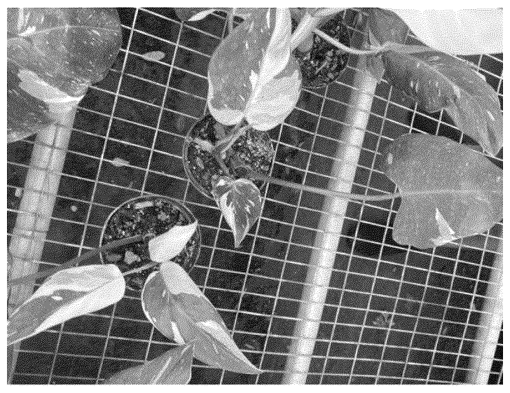

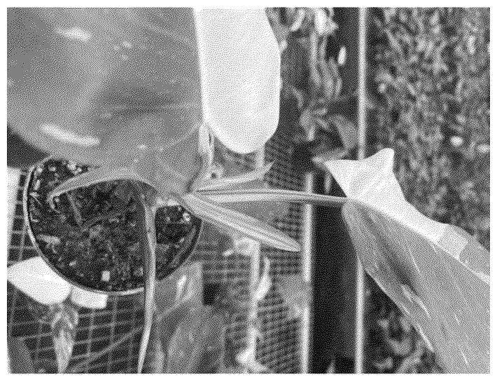

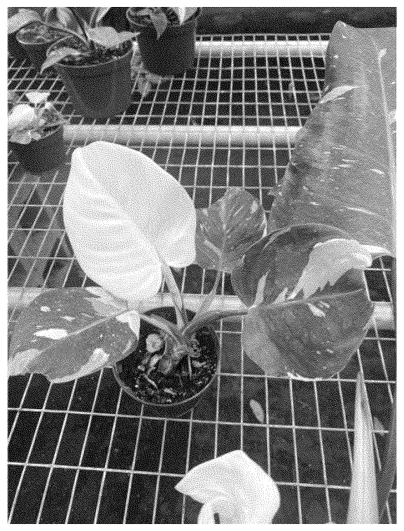

### BACKGROUND

The present invention relates to a new and distinct cultivar of Philodendron White Princess plant, botanically known as Araceae Philodendron White Princess, and hereinafter referred to by the cultivar name “White Ice Philodendron.”

The new Philodendron White Princess is a product of an accidental breeding by the Inventor in Umatilla, Florida.

The new Philodendron White Princess originated from an accidental breeding made by the Inventor in September 2020, of an unidentified selection of the Araceae Philodendron White Princess. The cultivar “White Ice Philodendron” was discovered and selected by the Inventor on September 2020, as a fully grown plant in an 8″ Growers Pot container within the progeny of the self-pollination in a controlled environment in Umatilla, Florida.

Compared to plants of the parent selection, plants of the new Philodendron White Princess are larger, branch earlier, are more freely branching, and have a less full plant habit and longer petioles.

Asexual propagation of the new Philodendron White Princess by taking cuttings at Umatilla, Florida has shown that the unique features of this new Philodendron White Princess plant are stable and reproduced true to type in successive generations.

### SUMMARY OF THE INVENTION

The new Philodendron White Princess has not been observed under all possible environmental conditions. The phenotype may vary somewhat with variations in environment such as temperature, light intensity, fertilizer rate, and/or irrigation amount and frequency without any variance in genotype.

The following traits have been repeatedly observed and are determined to be the unique characteristics of “White Ice Philodendron.” These characteristics in combination distinguish “White Ice Philodendron” as a new and distinct cultivar.
1. Plants of the new Philodendron White Princess are self-heading, not vining, and are self-supporting.
2. Plants of the new Philodendron White Princess are relatively small in size with a mostly upright, somewhat outwardly spreading and open plant habit.
3. Plants of the new Philodendron White Princess are vigorous and have a slow growth rate.
4. Plants of the new Philodendron White Princess branch quickly and are early branching.
5. Plants of the new Philodendron White Princess have large, thick, smooth, and flat leaves with pink, white, dark and light green leaves, pink with all white leaves, pink splash, and light and/or dark green leaves. Some leaves have a half-moon white color and some are all white.
6. Plants of the new Philodendron White Princess have relatively long petioles and long, green, pink, and white-colored petiole sheaths.

Plants of the new Philodendron White Princess can be compared to plants of the commercial Philodendron White Princess cultivars. However, in side-by-side comparisons conducted in Umatilla, Florida, plants of the new Philodendron White Princess differ from plants of the cultivars in the following characteristics:
1, Plants of the new Philodendron White Princess are larger and are more outwardly spreading and open than plants of the cultivars.
2. Plants of the new Philodendron White Princess are less vigorous, form branches earlier and are less freely branching than plants of the cultivars.
3. Leaves of plants of the new Philodendron White Princess are thicker, more ovate in shape with truncate bases, and have more numerous primary veins than leaves of plants of the cultivars.
4. Leaf color of plants of the new Philodendron White Princess is darker green and there is a more striking contrast between the mid rib and the surrounding leaf tissue compared to leaves of plants of the cultivars.
5. Plants of the new Philodendron White Princess have longer and wider petioles than plants of the cultivars. In addition, the shape of the petiole also differs longer, wider, and with green, white, and pink markings.
6. Leaf petiole sheaths of plants of the new Philodendron White Princess are greener and darker in color than leaf petiole sheaths of plants of the cultivars. Plants of the new Philodendron White Princess can also be compared to plants of the commercial Philodendron White Princess cultivars. However, in side-by-side comparisons conducted in Umatilla, Florida, plants of the new Philodendron White Princess differ from plants of the cultivars in the following characteristics:
1. Plants of the new Philodendron White Princess are larger and are more outwardly spreading and open than plants of the cultivars.
2. Plants of the new Philodendron White Princess are less vigorous, form branches earlier and are less freely branching than plants of the cultivars.
3. Leaves of plants of the new Philodendron White Princess are thicker, more ovate in shape with truncate bases and have more numerous primary veins than leaves of plants of the cultivars.
4. Leaf color of plants of the new Philodendron White Princess are white, and pink whereas leaf color of plants of the cultivars is green. Additionally, there is a more striking contrast between the midrib and the surrounding leaf tissue on plants of the new Philodendron White Princess compared to leaves of plants of the cultivars.
5. Plants of the new Philodendron White Princess have longer and wider petioles than plants of the cultivars. In addition, the shape of the petiole also differs.
6. Leaf petiole sheaths of plants of the new Philodendron White Princess are greener and lighter in color than leaf petiole sheaths of plants of the cultivars.
7. Plants of the new Philodendron White Princess flower later than plants of the cultivars.
8. Stems of plants of the new Philodendron White Princess are red, green, and/or pink and lighter in color than leaf petiole sheaths of plants of the cultivars. These stems are very long, round, and upright. These stems are medium sized diameter with vigorous roots and are slower to grow.

### Brief Description of the Photographs

The accompanying color photographs illustrate the overall appearance of the new Philodendron White Princess “White Ice Philodendron”, showing the colors as true as it is reasonably possible to obtain in color reproductions of this type.

The photographs on sheets one through four comprise top perspective views of a typical plant of “White Ice Philodendron” including immature leaves in a (size, e.g. 15 cm) container.

### DETAILED BOTANICAL DESCRIPTION

The following observations, measurements, and comparisons describe plants grown in Umatilla, Florida, under a polycarbonate-covered greenhouse and conditions which closely approximate those used in general, standard commercial horticultural practice. Plants were grown in fertilized soil under day temperatures ranging from 25 to 32 C and night temperatures ranging from 22 to 27 C. Light level ranged from 2,000 to 2,200 footcandles.

Plants used for this description were asexually reproduced by taking cuttings (twigs), rooted and planted into liquid and granular fertilized soil in 15-cm containers. Information was collected for this description and the cultivar comparisons in May, 2022, when the plants were about 2 months old from taking cuttings (twigs). Numerical measurements represent averages from typical plants. Environmental conditions were tropical, dry, and required less water than typical Philodendron White Princess.

In the following description, color references are made to The Royal Horticultural Society Colour Chart except where general terms of ordinary dictionary significance are used.

Botanical classification: Araceae Philodendron White Princess cultivar “White Ice Philodendron”.

Parentage: Self-pollination of an unidentified selection of Araceae Philodendron White Princess.

Propagation: Type.—By taking cuttings (twigs).

Time to initiate roots.—Summer: About 21 days at about 25 to 30° C. Winter: About 28 days at about 22 to 27° C.

Time to develop (Time to produce a rooted liner).—Summer: About 10 to 12 weeks at about 25 to 30° C. Winter: About 13 to 14 weeks at about 22 to 27 C.

Rooting habit.—Strong root system. Main roots thick; abundant lateral roots. Thick short aerial roots are occasionally observed.

Plant Description:

Growth habit.—Self-heading, not vining, and self-supporting. Mostly upright, but outwardly spreading, open, freely branching, Somewhat asymmetrical; mature plants, more rounded in overall shape. Appropriate for 15 to 25-cm containers.

Plant Size.—Height, Soil Surface to top of leaf canopy: About 50.7 cm. Width: About 99.2 cm.

Plant vigor.—Vigorous, slow growth rate.

Crop time.—About 48 to 52 weeks are required to produce a 40 to 50-cm tall plant in a 15-cm container from a 1 to 2-cm taken cutting (twigs).

Foliage description.—Shape of leaves on young plants: Between elliptic and ovate; apex, acuminate with some acute tendencies, base, obtuse; margin, entire. Shape of leaves on older plants: Ovate, apex, acuminate with Some acute tendencies, base, truncate; margin, entire. Length, mature leaves: About 36.7 cm. Width, mature leaves: About 18.8 cm. Length to width ratio, mature leaves: About 1.95 to 1. Aspect: Almost flat; Slightly concave at the petiole attachment. Texture: Thick, leathery, smooth. Surface: Glossy; young leaves very glossy. Color: Young, upper surface: At unrolling: Slightly darker than 144A. Once unrolled: Much darker than 144A. Young, lower Surface: 144A. Mature, upper surface: Darker than 147A. About 1 mm wide band along margin, lighter green, 144A. Mature, lower surface: Darker and Slightly greener than 144A, with subsequent development, leaf becomes slightly darker and greener than 147B. Venation, upper Surface of mature leaves: Midrib adjacent to petiole, 144B to 146B to 146C with short darker green striae; distal, darker green, 147A. Primary veins are slightly lighter green than surrounding leaf tissue. Venation, lower surface of mature leaves: midrib and primary veins, mostly 146C. Petiole aspect: Erect to semi erect. Petiole shape: Rounded lower Surface; upper Surface Slightly concave. Petiole length, primary shot: About 43.4 cm. Petiole diameter, just below leaf blade: About 10.3 mm. Petiole sheath length: About 21.2 cm. Petiole color, young leaves:144A to 146C with short darker green striae. Petiole color, upper Surface of mature leaves: Adjacent to leaf blade, close to 146A to 146B with Some 144A, proximally darker to 146A to 147B; slightly lighter near base. Short darker green Striae. Petiole color, lower Surface of mature leaves: Adjacent to leaf blade, close to 146B to 146D and 14.4A to 144B; proximally darker, close to 146A, base, close to 144A. Short darker green striae. Petiole sheath:144B and 144D with short darker green striae, Some grayed orange, close to 166C to 166D, present at proximal end near edges, more noticeable at distal end.

Inflorescence.—Typical of Philodendron White Princess, no commercial significance. Plants typically form inflorescences about 18 to 23 months after planting of a twig taken from cutting. Description of inflorescences taken from plants grown for about 2.5 years after planting a twig taken from cutting.

Arrangement.—Spathes with spadices with one to three inflorescences in each floral sympodium. Each developing inflorescence enclosed by two sheaths, which deteriorate before spathe opens. Spathes erect, situated near shoot base on short, thick, usually bent peduncles. Spathes typically tightly tolled around spadices, Spathes typically open for about one day. When spathes are open, the spadix is fully exposed, leaning forward beyond spathe margins, at about 50 to penduncle axis.

Fragrance.—weak, Similar to other Philodendrons, but weaker.

Peduncle.—Length: About 7.3 cm. Diameter: About 1.5 cm. Color: 144B or between 144B and 144C.

Spathe.-Shape: Boat-shaped; longitudinal section elliptic; apex between acuminate and cuspidate; base, obtuse; margin, entire and usually rolled back especially distally. Length: About 18.3 cm. Width: About 7.6 cm. Length to width ratio: About 2.4 to 1. Depth: About 4.8 cm. Texture: Very thick, fleshy, not glossy. Color: Open, front surface: Between 47A and 47B around spadix base, distally and toward margin, much lighter, 48D to 49D to close to 159C near apex with some scattered light pink. Some yellow green, 144B, on midrib and along margins near apex. Open, rear surface: Most of the central Zone of proximal half is an uneven color, varying between different combinations of 146C, 146D, 144A, 144B and 144C. Color becomes lighter and more yellow towards margins, near margins, between 145C and 145D and translucent. In the distal half, there is a band along midrib between 144B, and 146C and 146D. Spathe color on either side of this band is variable, but much lighter and more yellow, most areas more yellow than 145C and more green than 150C. Often there is a wide, almost whitish, translucent band on one side near the margin.

Spadix.—Shape: Cylindrical, Strongly tapering towards acute apex. Female Zone, Situated at the base, shorter than the male Zone and Separated by an intermediate Sterile Zone. Sterile and male Zones not clearly distinguished when Spathe is open. Spadices typically sessile. Length: About 17.1 cm; female Zone, about 4.8 cm; Sterile and male Zones combined, about 12.6 cm. Diameter: Female Zone, about 1.9 cm; sterile and male zones, about 1.8 cm proximally. Color: Female Zone, between 145C and 145D; sterile and male zones, slightly darker than 159C. Pollen: Cream in color. Dehiscence about one to three days after spathe closes.

Seed production.—Seed production has not been observed. Typically spathes and spadices gradually deteriorate after spathe closure.

Disease tolerance: Resistance to pathogens common to Philodendron White Princess has not been observed.

What is claimed is:

1. A new and distinct cultivar of Philodendron White Princess plant named “White Ice Philodendron”, as illustrated and described.
